(19) United States
(12) Patent Application Publication
(10) Pub. No.: US 2020/0386670 A1
(43) Pub. Date: Dec. 10, 2020

(54) COLOR MEASUREMENTS OF TURBID LIQUIDS

(71) Applicants: CHR. HANSEN NATURAL COLORS A/S, Hoersholm (DK); UNIVERSITE MONTPELLIER, Montpellier (FR); CENTRE NATIONAL DE RECHERCHE SCIENTIFIQUE, Paris cedex 16 (FR)

(72) Inventors: Frederic GENIET, Montpellier (FR); Stephanie VUILLEMOT-KESENNE, Arpajon (FR); Maeva GARDON-CHAPELET, Arpajon (FR)

(73) Assignees: CHR. HANSEN NATURAL COLORS A/S, Hoersholm (DK); UNIVERSITE MONTPELLIER, Montpellier (FR); CENTRE NATIONAL DE RECHERCHE SCIENTIFIQUE, Paris cedex 16 (FR)

(21) Appl. No.: 16/959,636

(22) PCT Filed: Dec. 20, 2018
(86) PCT No.: PCT/EP2018/086345

§ 371 Date: Jul. 1, 2020

(30) Foreign Application Priority Data
Jan. 4, 2018 (EP) 18150259.2

(51) Int. Cl. G01N 21/25; G01N 33/14; G01N 33/04; G01N 33/03

(52) U.S. Cl. CPC G01N 21/251; G01N 33/14; G01N 2201/08; G01N 33/03; G01N 33/04

(57) ABSTRACT

The present invention relates to a set-up and a method for measuring color of a turbid liquid giving a result matching a visual observation.

### FIELD OF THE INVENTION

The present invention relates to the field of color measurements of turbid liquids.

### BACKGROUND

Color measurement systems help to improve the operational efficiency and product quality in supply chains. For example, within the food industry and specifically beverage, confectionary, dairy and prepared food manufactures relying on the digital color workflow require accurate color evaluation and visualization.

Within many industries it is important to get the color of the product right during production and to ensure stability of the color during storage both before usage and when added to other products. For transparent liquids it is easy to measure the color but the color of cloudy turbid liquids which are scattering the light cannot be measured accurately because the back scatter from the turbidity interferes with the measurement. Existing methods for measuring the color of liquids include the use of spectrocolorimeter which gives good results for transparent solutions, but it is not efficient for cloudy/turbid solutions as it gives a color reading which resembles a much darker color than the actual visually observed color.

WO2002075285 relates to an apparatus for measuring the color of wet paints, so the color of a material being made can be accurately matched to a standard color in the wet state with confidence that the color will match in the dry state. The color is being measured when the wet paint is flowing through the apparatus.

No good and reliable methods for measuring the color of turbid liquids are known today.

Visual color evaluation is known to be used. In this case the sample is compared to series of color standards in order to see which standard the sample most closely resembles. This method is only approximate, and the downside is that the evaluation may differ from one person to the other and cannot be replicated. Others recommend diluting the turbid liquid sample until transparency; however it will change the color. Others again recommend removing the particles in the turbid liquid making the liquid transparent by filtering or centrifugation, but it alters both the product and the color.

Therefore there is a need for a simple measurement method which can measure the color of a turbid liquid and give reliable results matching a visual observation. In particular there is a need for a simple set up for measuring the color of a final product i.e. a turbid beverage in its final packaging such as a bottle without changing the product by dilution or removal of product.

### SUMMARY OF THE INVENTION

The inventors of the present invention have surprisingly invented a set-up and a measuring method where it is possible to measure the color of a finished product ready to be commercialized comprising a turbid liquid which matches a visual observation as the customer will experience it in the store, without destroying the product during the measurement.

The object of the present invention is to provide a set-up and a method for measuring color of a turbid liquid giving a result matching a visual observation.

A first aspect of the present invention relates to a set-up for measuring a color of a turbid liquid to obtain a result matching a visual color observation comprising:
i) a housing wherein at least 50% of the inside area is white; and
ii) a light source mimicking daylight illuminating the inside of the housing giving consistent light conditions;
iii) a spectrophotometer;
iv) a transparent sample holder, comprising a turbid liquid, placed inside the housing positioned against the optical fiber connected to the entrance slit of the detector of the spectrophotometer;
v) a computer; and
vi) a spectrophotometric software which is able to collect and calculate colorimetric data such as lightness, chroma and hue.

A second aspect of the present invention relates to a set-up for measuring a color of a turbid liquid comprising:
i) a housing wherein at least 50% of the inside area is white; and
ii) a light source mimicking daylight illuminating the inside of the housing giving consistent light conditions;
iii) a spectrophotometer
iv) a transparent sample holder comprising a turbid liquid which is stationary in the holder, wherein the sample holder is positioned against the optical fiber connected to the entrance slit of the detector of the spectrophotometer of iii);
v) a computer; and
vi) a spectrophotometric software which is able to collect and calculate colorimetric data being lightness, chroma and hue.

A second aspect of the present invention relates to a method for measuring a color of a turbid liquid to obtain a color result in the form of colorimetric values such as chroma, hue and lightness matching a visual color observation comprising the steps of:
i) Providing a housing with
   a) at least 50% of the inside area of the housing is white;
   b) with a light source mimicking daylight;
   c) a spectrophotometer; and
   d) a transparent sample holder comprising a turbid liquid color sample;
ii) illuminating the turbid liquid sample of i)d) with the use of the light source of i) b);
iii) measuring the color spectrum of the turbid liquid sample;
iv) converting the spectrum of iii) into spectrocolorimetric values using a computer and a software for doing so; and
v) providing a digital color read out in the form of colorimetric values such as chroma, hue and lightness.

A third aspect of the present invention relates to a method for measuring a color of a turbid liquid to obtain a color result in the form of colorimetric values such as chroma, hue and lightness matching a visual color observation comprising the steps of:
i) Providing a housing with
   a) at least 50% of the inside area of the housing is white;
   b) with a light source mimicking daylight;
   c) a spectrophotometer; and
   d) a transparent sample holder comprising a turbid liquid color sample;
ii) illuminating the turbid liquid sample of i) d) with the use of the light source of i) b);
iii) measuring the color spectrum of the turbid liquid sample;
iv) converting the spectrum of iii) into spectrocolorimetric values using a computer and a software for doing so; and
v) providing a digital color read out in the form of colorimetric values such as chroma, hue and lightness corresponding to a visual observation.

A fourth aspect of the present invention relates to the above measurement method wherein the sample to be measured is a turbid liquid, but transparent liquids may also be measured by it, any colored liquids, but the method can also be used for other food products such as confectionary, dairy products and prepared foods. The method is suitable for all applications that are diffusing and scattering the light.

### BRIEF DESCRIPTION OF THE DRAWINGS

The above, as well as additional objects, features and advantages of the present invention, will be better understood through the following illustrative and non-limiting detailed description of preferred embodiments of the present invention, with reference to the appended drawings, wherein:

FIG. 1 shows virtual colors corresponding to colorimetric values measured with existing colorimeter Datacolor 650 on samples with increasing turbidity having the same visual color.

FIG. 2 shows virtual colors corresponding to colorimetric values measured with the present invention on samples with increasing turbidity having the same visual color.

FIG. 3 shows a drawing of a possible setup of the present invention.

### DETAILED DESCRIPTION

The objective of the present invention is to obtain colorimetric values representative of visual observations of turbid liquids. The inventors have surprisingly found that by illuminating a turbid liquid sample contained in a transparent sample holder present in a white colored housing with an illuminant resembling daylight, it is possible by a direct measurement by a spectrophotometer on the sample present in the transparent sample holder to obtain a spectrum which is converted into L*C*h values via a suitable software, and said values when shown as a read-out color is matching what can visually be observed.

The set-up and method of the present invention provides a set-up designed to achieve accurate color measurement of turbid liquids. The set-up and method described provide a simple solution that enables technicians or other technical professionals to obtain more accurate color measurement values of turbid liquids.

The method using the set-up of the invention is a very convenient, simple and reliable method for measuring the color of turbid liquids typically found within the food industry in particular within the beverages industry more particular within the juice, soft drinks and alcoholic drinks industry.

The present invention relates to a set-up for measuring a color of a turbid liquid to obtain a result matching a visual color observation comprising:
i) a housing wherein at least 50% of the inside area is white;
ii) a light source mimicking daylight illuminating the inside of the housing giving consistent, light conditions;
iii) a transparent sample holder placed inside the housing positioned against the optical fiber connected to the entrance slit of the detector of the spectrophotometer;
iv) a colored turbid liquid to be poured into the sample holder of iii);
v) a spectrophotometer;
vi) a computer; and
vii) a spectrophotometric software which is able to collect and calculate colorimetric data such as lightness, chroma and hue.

The present invention also relates to a set-up for measuring a color of a turbid liquid comprising:
i) a housing wherein at least 50% of the inside area is white; and
ii) a light source mimicking daylight illuminating the inside of the housing giving consistent light conditions;
iii) a spectrophotometer
iv) a transparent sample holder comprising a turbid liquid which is stationary in the holder, wherein the sample holder is positioned against the optical fiber connected to the entrance slit of the detector of the spectrophotometer of iii);
v) a computer; and
vi) a spectrophotometric software which is able to collect and calculate colorimetric data being lightness, chroma and hue.

The present invention further relates to a method for measuring the color of a turbid liquid matching a visual color observation comprising the steps of:
i) Providing a housing with
   a) at least 50% of the inside area is white;
   b) a light source;
   c) a spectrophotometer; and
   d) a transparent sample holder comprising a turbid liquid color sample;
ii) illuminating the turbid liquid color sample of i) d) with the use of the light source of i) b);
iii) measuring the color spectrum of the turbid liquid sample with the spectrophotometer;
iv) converting the spectrum of iii) into colorimetric values using a computer and a software for doing so; and
v) providing a digital color read out corresponding to a visual observation.

The invention also relates to a method for measuring the color of a final product comprising a turbid liquid matching a visual color observation comprising the steps of:
i) Providing a housing with
   a) at least 50% of the inside area is white;
   b) a light source;
   c) a spectrophotometer; and
   d) a final product comprising a turbid liquid;
iii) illuminating the turbid liquid of i) d) with the use of the light source of i) b);
iv) measuring the color spectrum of the turbid liquid i) d) of with the spectrophotometer;
v) converting the spectrum of iv) into colorimetric values using a computer and a software for doing so; and
vi) providing a digital color read out corresponding to a visual observation.

Digital color is given by the software depending on the lightness, intensity and shade calculated as CIE colorimetric values from the luminous flux received by the spectrophotometer after the light is diffused on the sample. The visual comparison between the digital color of the software and the visual observation can be done directly.

Turbidity is defined in the present invention as an optical property of a semitransparent material for which light gets scattered and absorbed instead of traveling through the liquid in a straight line, making the liquid look cloudy. In other words, turbidity, in most cases, is a measure of relative sample clarity. Color and turbidity are two different properties of a liquid. Turbidity is due to light scattering, whereas color is developed due to absorption of light. Without being bound by theory it is believed that the presence of undissolved particles larger than 0.2 μm in a liquid makes it turbid. The undissolved particles scatter a beam of light when passed through the liquid. This causes the attenuation of the intensity of the light beam. The ratio of the intensity of the incident and transmitted light is proportional to the amount of undissolved particles present in the liquid or the turbidity of the liquid. This light scattering influences the measurement of the color and instead of giving a result resembling the actual color, the existing color measurement method will give a result which resembles a much darker color depending on the level of turbidity.

It has been found that when measuring on liquids with a turbidity above 25 NTU (Nephelometric Turbidity Unit), known colorimetric methods are not reliable, whereas the present invention gives results matching visible observations. Turbidity is measured by an instrument called a nephelometer or a turbiditymeter.

For example the transmission of light through fruit concentrates is less than 10% which gives a color reading on a spectrocolorimeter resembling a dark and murky color even though the color might be bright yellow.

The liquid of the present invention is characterized as being turbid, which for the purposes herein shall mean that it appears semi-opaque or opaque due to its cloudiness. In a particular embodiment of the present invention the turbid liquid has a turbidity above 25 NTU. In a more particular embodiment of the present invention the turbid liquid has an NTU above 30. the invention does not measure the turbidity of the liquid, but the method and its set-up avoid that turbidity disturbs the measurement, as it happens with the existing color measurements methods.

The set-up and method of the present invention is particularly useful for measuring the color of turbid liquids. The turbid liquid is preferably selected from the group consisting of but not limited to food products such as dairy products, oil products such as emulsions including mayonnaise and salad dressings, beverages such as fruit juice products, smoothies, soft drinks, alcoholic drinks such as wine and beer, and liquid cosmetic products such as skin lotions, skin tonics, shampoos, soaps etc., where it is useful to measure color and obtain reliable color readings resembling visual observations of a turbid liquid. The liquid may contain live microorganisms, such as yeasts or lactic acid bacteria or both, an example being various fermented milk products and whey, unfiltered beer and other opaque fermented beverages. The liquid may further comprise solids, such as suspensions and dispersions.

In a particular embodiment of the present invention the turbid liquid is an undiluted final product, such as a beverage.

The transparent sample holder wherein the turbid liquid sample is placed within the housing may be in any form. In a particular embodiment the sample holder is transparent and colorless in order not to interfere with the color measurement. By colorless is meant, that the material of the sample holder does not comprise any color which may interfere with the color measurements. The sample holder may be of any kind of suitable material. In a particular embodiment of the present invention the sample holder may be made of glass or of plastic. The sample holder may be selected from but is not limited to a tube, a bottle, a beaker or a cuvette. In a particular embodiment the sample holder is a bottle. If comparison of two samples is needed, it is necessary to use the same or identical sample holder. To obtain the most authentic result, ideally the sample holder and volume of the sample should be identical to the final product. In a particular embodiment the sample holder is the packaging of the final product, i.e. a drinking bottle.

The housing used in the present invention may have any form such as a cylinder, a cube, a pyramid or a sphere. The inside of the housing should be able to reflect as much light as possible in order for the spectrophotometer to receive as much information as possible for obtaining a measurement value identical to or matching the visual color. In order to reflect as much light as possible a large part of the inside area of the housing should be white, as the color white reflects all colors of light.

In a particular embodiment of the present invention at least 50%, such as at least 60%, such as at least 75% of the inside area of the housing is white. In a more particular embodiment at least 80% of the inside area of the housing is white. In a more particular embodiment of the present invention at least 85% of the inside area of the housing is white, more particular at least 90% such as at least 95%.

The housing should be large enough to be able to comprise a spectrophotometer, a sample holder or a final product such as a soft drink bottle and a light source. In a particular embodiment of the present invention the housing is a light cabinet.

There are several ways to classify the color of a substance. Generally the color characteristics of a substance may be characterized by three parameters, the hue, the saturation and the lightness. Hue is an attribute associated with each of the dominant wavelengths of the visible spectrum and reflects the dominant color of the composition (red, yellow, blue etc.). Chroma is the saturation and pertains to the intensity of the color composition often described as the vividness or the dullness of a color. Lightness reflects the amount of white or black in the color composition.

A number of scales exist to measure color. The earliest developed scale is the Munsell system. This system assigns numerical values to the three properties of colors, namely Chroma, Hue and Lightness. The Munsell scale was developed and based upon human perception of color. Modern day color measurements utilize instruments such as colorimeters and spectrophotometers. The instruments measure spectral data across the visible spectrum.

In a particular embodiment of the present invention the set-up comprises the use of a spectrophotometer or a similar instrument capable of taking readings on hue, chroma and lightness in accordance with CIE (Commission Internationale de L′Eclairage—the International Commission of Illumination) tristimulus system: CIELCH. In CIELCH system, L* denotes lightness, C* specifies chroma and h denotes the hue.

The CIE Color System is based on numerical descriptions of light (or illuminant), object and observer.

CIE XYZ system calculates tristimulus values that are computed wavelength by wavelength, by multiplying illuminant, object, & observer data, at each wavelength, then adding products. CIELCH system, corresponding to the L*C*h color space, similar to CIELAB (Lab systems describe colors in terms of red/green and yellow/blue components), is preferred by some industry professionals because its system correlates well with how the human eye perceives color. It has the same diagram as the L*a*b* color space but uses cylindrical coordinates instead of rectangular coordinates. Hue is calculated from a* (red-green axe) and b* (yellow-blue axe).

The present method is not depending on any particular color expression system and therefore any present or future color expression system may also apply in the method of the present invention. An extensive description of the various color representation parameters can be found in the pamphlet: Minolta: Precise Color Communication, “color control from feeling to instrumentation” Minolta CO., Ltd, 1994, which is hereby incorporated by reference.

In a particular embodiment of the present invention the color is measured by a spectrophotometer such as Probe4light from Pleiades Instruments or any other suitable spectrophotometer, and the spectrum is converted by a suitable software such as PhotonColor from Majantys or any suitable software available to calculate colorimetric values, software which can give the results of L, C and H and the DE 2000 value.

DE2000 is a value to represent the difference between 2 measured samples. It is calculated from the values L* (lightness), C* (Chroma) and h.

The spectrophotometer is used to acquire colorimetric data associated with the display on the computer monitor or similar display device. The spectrophotometer is positioned inside the housing. The optical fiber connected to the entrance slit of the detector of the spectrometer should be up against the sample holder, in order for the spectrometer to measure directly on the sample holder. The color measurement performed by the spectrophotometer according to the present invention is non-destructive: a direct measurement on the sample.

Suppliers of spectrophotometers include XRITE, DataColor, Konica Minolta, BYK Gardner, HunterLab.

Calibration of the spectrophotometer is necessary before color shade measurement in order to define a black and a white reference.

A spectrophotometer is a measuring apparatus decomposing a light beam into a spectrum. The variable used is the light intensity; the measured quantity is the wavelength.

The optical fiber connected to the entrance slit of the detector, where the spectrum is projected, receives a sum of data for each wavelength. If the sum of data is small, it means that part of the spectrum had been absorbed by the sample measured. It is then possible to infer/deduce the color shade of the sample. In other words, it is possible to infer/deduce from spectrum absorption or transmittance data that will be calculated as colorimetric details.

During measurement the sample is exposed to a continuous light exposure. The continuous light exposure ensures sufficient light energy to restore the complete color spectrum. The color spectrum is calculated and converted into colorimetric value in CIELCH system giving lightness L*, chroma C* and hue h. The color read out seen on the computer is showing the same color as observed by visual observation of the sample placed in the housing.

Colorimetric data can be used as such and compared with another sample to measure the color difference. Colorimetric data can also be recorded over time in the framework of an aging test. Samples over time evolution can be measured and data after aging can be compared with the data before aging in order to evaluate the robustness of the product.

In a particular embodiment of the present invention the light source used should be mimicking natural daylight, as color or shade looks different when viewed under different light conditions. The difference in color may be extreme and obvious, but in a supply chain, even minor differences in perceived color can be problematic. Color accuracy is a major concern for manufactures asked to supply liquid products such as beverages in very precise shades.

The light source used should therefore preferably mimic daylight. The industry standard for daylight is D65.

In a particular embodiment of the present invention the light source is D65 that is standard illuminant defined by the International Commission on Illumination (CIE) for the industry for various applications with a correlated color temperature of 6504K; described and referenced in ISO: 3668, ASTM 1729 and DIN6173-2. D65 is conforming highly to the CIE specifications, for accurate color matching.

D65 corresponds roughly to the average midday light in Western Europe/Northern Europe (comprising both direct sunlight and the light diffused by a clear sky), hence it is also called a daylight illuminant. CIE standard illuminant D65 should be used in all colorimetric calculations requiring representative daylight, unless there are specific reasons for using a different illuminant. Variations in the relative spectral power distribution of daylight are known to occur, particularly in the ultraviolet spectral region, as a function of season, time of day, and geographic location. The light source may be placed anywhere within the housing as long as it is not reflecting its light in the sample holder giving disturbance in the color measurement.

The light source should not illuminate the input fiber of the spectrophotometer directly. Ideally the sample holder will be enlightened by diffuse light coming from the white walls only.

In a particular embodiment the light source is placed above the sample.

The set-up and the measurement method may be used in any industry where the measurement of the color of turbid liquids are required such as within the beverage, the confectionary, the dairy and the prepared food industries.

In a particular embodiment of the present invention the method and the set-up is for measuring the color on products selected from the group consisting of but not limited to food products such as dairy products, oil products such as emulsions including mayonnaise and salad dressings, beverages such as fruit juice products, smoothies, soft drinks and alcoholic drinks such as wine and beer, and liquid cosmetic products such as skin lotions, skin tonics, shampoos, soaps etc. In a particular embodiment the color is measured on a beverage such as soft drinks, beer, juice and/or smoothies.

An advantage of the present invention is that it is possible to measure directly on the final product to be sold to the customer i.e. a beverage in a transparent and colorless bottle. Whereby the measurement can give a digital color readout corresponding to the visual observation of the customer in the store.

The method of the present invention is a nondestructive method meaning that the sample measured on is not being destroyed during the color measurement. Further no cleaning is necessary after measurement and it is a non-destructive method.

ITEMS
1. A set-up for measuring a color of a turbid liquid to obtain a result matching a visual color observation comprising:
   i) a housing wherein at least 50% of the inside area is white; and
   ii) a light source illuminating the inside of the housing giving consistent, light conditions;
   iii) a transparent sample holder placed inside the housing positioned against the optical fiber connected to the entrance slit of the detector of a spectrophotometer;
   iv) a colored turbid liquid to be poured into the sample holder of iii);
   v) a spectrophotometer for measuring the color spectrum of the colored turbid liquid;
   vi) a computer; and
   vii) a spectrophotometric software which is able to collect and calculate colorimetric data.
2. A set-up for measuring a color of a turbid liquid comprising:
   i) a housing wherein at least 50% of the inside area is white; and
   ii) a light source mimicking daylight illuminating the inside of the housing giving consistent light conditions;
   iii) a spectrophotometer
   iv) a transparent sample holder comprising a turbid liquid which is stationary, wherein the sample holder is positioned against the optical fiber connected to the entrance slit of the detector of the spectrophotometer of iii);
   v) a computer; and
   vi) a spectrophotometric software which is able to collect and calculate colorimetric data being lightness, chroma and hue.
3. The set-up according to any of the preceding items, wherein the colorimetric data are lightness, chroma and hue.
4. The set-up according to any of the preceding items, wherein the light source of ii) is mimicking daylight.
5. The set-up according to any of the preceding items, wherein at least 65% of the inside area is colored white.
6. The set-up according to any of the preceding items, wherein at least 75% of the inside area is colored white.
7. The set-up according to any of the preceding items, wherein the sample holder is colorless.
8. The set-up according to any of the preceding items, wherein the light is positioned above the sample.
9. The set-up according to any of the preceding items, wherein the sample holder is placed in the middle of the housing.
10. The set up according to any of the preceding items, wherein the spectrophotometer is placed within the housing.
11. The set up according to any of the preceding items, wherein the light source is placed within the housing.
12. The set-up of any preceding items, wherein the sample holder is removable.
13. The set-up according to any preceding items, wherein the sample holder is the packaging of a final product.
14. The set-up according to any preceding items, wherein the turbid liquid is a non-diluted final product.
15. The set-up according to any of the preceding items, wherein the housing is a light cabinet.
16. The set-up according to any of the preceding items, wherein the method is non-destructive.
17. The set-up according to any of the preceding items, wherein the observed color is similar to what the consumer would observe.
18. A method for measuring the color of a turbid liquid matching a visual color observation comprising the steps of:
   i) Providing a housing with
      a) at least 50% of the inside area is white;
      b) a light source;
      c) a spectrophotometer; and
      d) a transparent sample holder comprising a turbid liquid color sample;
   ii) illuminating the turbid liquid color sample of i) d) with the use of the light source of i) b);
   iii) measuring the color spectrum of the turbid liquid sample with the spectrophotometer;
   iv) converting the spectrum of iii) into colorimetric values using a computer and a software for doing so; and
   v) providing a digital color read out.
19. A method for measuring the color of a final product comprising a turbid liquid matching a visual color observation comprising the steps of:
   i) Providing a housing with
      a) at least 50% of the inside area is white;
      b) a light source;
      c) a spectrophotometer; and
      d) a final product comprising a turbid liquid;
   iii) illuminating the turbid liquid of i) d) with the use of the light source of i) b);
   iv) measuring the color spectrum of the turbid liquid i) d) of with the spectrophotometer;
   v) converting the spectrum of iv) into colorimetric values using a computer and a software for doing so; and
   vi) providing a digital color read out.
20. A method for measuring the color of a turbid liquid matching a visual color observation comprising the steps of:
   i) Providing a housing with
      a) at least 50% of the inside area is white;
      b) a light source;
      c) a spectrophotometer; and
      d) a transparent sample holder comprising a turbid liquid color sample;
   ii) illuminating the turbid liquid color sample of i) d) with the use of the light source of i) b);
   iii) measuring the color spectrum of the turbid liquid sample with the spectrophotometer;
   iv) converting the spectrum of iii) into colorimetric values using a computer and a software for doing so; and
   v) providing a digital color read out corresponding to
      a) a visual observation of the color of the turbid liquid or
      b) the color of the turbid liquid.
21. A method for measuring the color of a final product comprising a turbid liquid matching a visual color observation comprising the steps of:
   i) Providing a housing with
      a) at least 50% of the inside area is white;
      b) a light source;
      c) a spectrophotometer; and
      d) a final product comprising a turbid liquid;
   iii) illuminating the turbid liquid of i) d) with the use of the light source of i) b);
   iv) measuring the color spectrum of the turbid liquid i) d) of with the spectrophotometer;
   v) converting the spectrum of iv) into colorimetric values using a computer and a software for doing so; and
   vi) providing a digital color read out corresponding to
      a) a visual observation of the color of the turbid liquid, or
      b) the color of the turbid liquid.
22. The method of any of items 18 to 21, wherein the final product is a beverage placed in transparent bottle.
23. The method of any of items 18 to 22, for measuring the color of a turbid liquid in the form of chroma, hue and lightness comprising the steps of:
   i) Providing a housing with
      a) at least 50% of the inside area is white;
      b) a light source mimicking daylight;
      c) a spectrophotometer; and
      d) a transparent sample holder comprising a turbid liquid color sample;
   ii) illuminating the turbid liquid sample of ii) with the use of the light source of i) c);
   iii) measuring the color spectrum of the turbid liquid sample with the spectrophotometer;
   iv) converting the spectrum of iv) into spectrocolorimetric values using a computer and a software for doing so; and
   v) providing a digital color read out in the form of chroma, hue and lightness corresponding to
      a) a visual observation of the color of the turbid liquid, or
      b) the color of the turbid liquid.
24. The method according to any of items 18 to 23, wherein at least 65% of the inside area is white.
25. The method according to any of items 18 to 24, wherein at least 75% of the inside area is white.
26. The method according to any of the items 18 to 25, wherein the transparent sample holder is the packaging of a final product.
27. The method according to any of items 18 to 26, wherein the transparent sample holder comprising the turbid liquid color sample is a final product.
28. The method according to any of items 18 to 27, wherein the liquid is stationary.
29. The method according to any of items 18 to 28, wherein the liquid is not flowing.
30. The method according to any of items 18 to 29, for measuring the color of food products, paints, hair products, body care products and/or cosmetics.
31. The method according to item 30 wherein the food product is selected from a dairy product, an oil product and/or a beverage.
32. The method according to item 31, wherein the beverage is selected from fruit juice products, smoothies, soft drinks and/or alcoholic drinks.
33. The method of item 30, wherein the body care products are selected from skin lotion, skin tonic, shampoo and/or soap.
34. Use of the set up according to any of the items 1 to 17, for measuring a color of a turbid liquid to obtain a result matching a visual color observation.

### EXAMPLES

Set-up for measuring color of turbid solutions.

Equipment:

Light Cabinet white colored on the inside—Light Cabinet Verivide:

Model: CAC 120 with dimensions (mm) Width/Height/Depth: 1290/755/620 (Overall) or 1260/570/585 (Viewing Area).

Spectrophotometer:

Model Probe4Light 32 bits, Pleiades Instruments, originally commercialized by Majantys.

Software to calculate spectrum data as colorimetric values:

Software: PhotonColor, Pleiades Instruments originally by Majantys.

The software must be configured as follows in order to obtain the calculated colorimetric values before proceeding with measurements:
Iluminant: D65
Type observer: 10°
Type of diagram: CIE 1964
Spectrum: 380 to 780 nm
Resolution: 128 pixels—“loop” value: 1
Number of flashes: 1
Integration time (duration of 1 flash): 1000 ms
Flash: continuous mode

If equipment and/or set-up vary, it might be necessary to adapt the parameters. For example, spectrophotometer can be saturated if the luminous flux received is too high. It might be necessary to reduce the integration time.

### Example 1

### Color Measurement of a Liquid with Yellow Color

Application in a standard soft-drink (11.0° B—pH=3.0 made of saccharose, citric acid, potassium sorbate and sodium benzoate as preservatives) of a yellow transparent emulsion (ColorFruit® Yellow 010 WSS) at 0.3 g/L drink. A cloudifier (white emulsion used to increase the cloudiness) was applied gradually from 0 to 1.5 g/L drink, see table 1.

TABLE 1
Concentration of cloudifier vs. measured turbidity.

| Concentration of cloudifier | Turbidity |
|---|---|
| (g/L) | (NTU) |
| 0 | 0.54 |
| 0.1 | 20 |
| 0.2 | 41 |
| 0.3 | 61.2 |
| 0.4 | 85.6 |
| 0.5 | 112 |
| 0.6 | 140 |
| 0.7 | 167.7 |
| 0.8 | 199.3 |
| 0.9 | 226.1 |
| 1 | 262 |
| 1.5 | 419 |

Color was measured with the two methods 1-A and 1-B:

1-A Colorimetric values measured with existing spectrocolorimeter Datacolor 650:

TABLE 2
Colorimetric values measured with Datacolor 650.

| Concentration of cloudifier | | | |
|---|---|---|---|
| (g/L) | L* | C* | h |
| 0 | 83.56 | 109.63 | 85.41 |
| 0.1 | 77.28 | 109.21 | 84.04 |
| 0.2 | 70.99 | 106.18 | 82.89 |
| 0.3 | 65.77 | 100.83 | 82.03 |
| 0.4 | 60.59 | 95.52 | 81.07 |
| 0.5 | 55.22 | 89.31 | 79.95 |
| 0.6 | 50.77 | 83.23 | 79.08 |
| 0.7 | 46.31 | 77.1 | 78.03 |
| 0.8 | 41.35 | 70.25 | 76.77 |
| 0.9 | 38.38 | 65.41 | 76.21 |
| 1 | 34.24 | 59.13 | 74.86 |
| 1.5 | 21.88 | 39.09 | 71.28 |

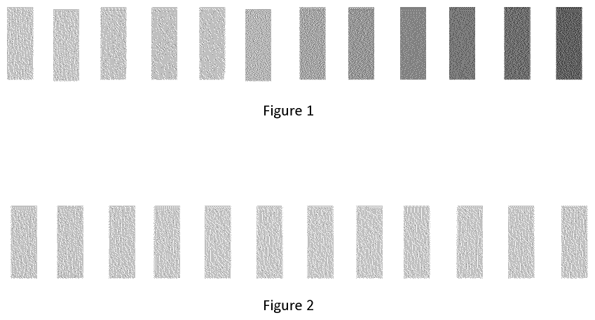

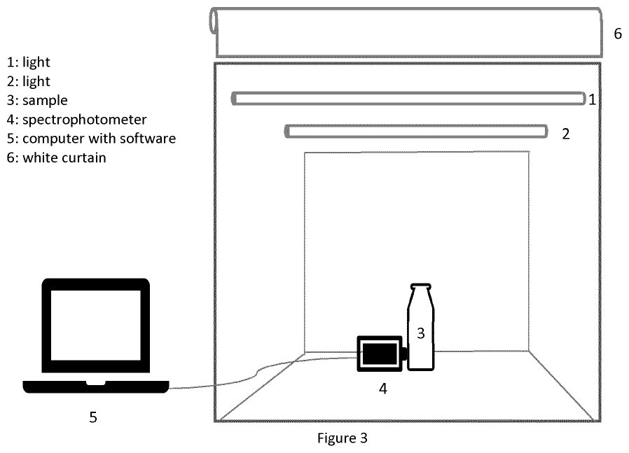

Also shown in FIG. 1.

1-B Colorimetric values measured according to the present invention:

TABLE 3
Colorimetric values measured according to the invention.

| Concentration of | | | | |
|---|---|---|---|---|
| cloudifier (g/L) | L* | C* | h | Turbidity |
| 0 | 89.74 | 126.11 | 89.29 | 0.54 |
| 0.1 | 91.04 | 127.36 | 89.26 | 20 |
| 0.2 | 91.58 | 127.80 | 89.28 | 41 |
| 0.3 | 91.83 | 127.92 | 89.12 | 61.2 |
| 0.4 | 92.09 | 128.98 | 88.97 | 85.6 |
| 0.5 | 91.56 | 128.77 | 88.71 | 112 |
| 0.6 | 91.39 | 127.42 | 88.45 | 140 |
| 0.7 | 91.03 | 127.34 | 88.35 | 167.7 |
| 0.8 | 90.88 | 125.71 | 88.19 | 199.3 |
| 0.9 | 90.68 | 125.99 | 88.19 | 226.1 |
| 1 | 90.12 | 123.81 | 87.79 | 262 |
| 1.5 | 88.26 | 121.17 | 87.34 | 419 |

Also shown in FIG. 2.

All 3 values L*, C* and h obtained with the old method were significantly lower than with the new method. It was obvious from 20 NTU (0.2 g/L cloudifier): 20.59 points difference with the lightness and 21.62 with the chroma, 6.39 with the hue, the virtual shade turned to a darker, duller and more orange color than the yellow color of the sample. With 0.6 g/L cloudifier, the virtual color obtained with the old method appeared brownish whereas the observed color was yellow.

The virtual color, calculated from the colorimetric values, given by the software of the old method, is systematically more dull and darker than the color visually observed on the sample measured on.

The color read out from the new set-up showed the same color as visually observed on the sample measured on.

### Example 2

### Color Measurement of a Liquid with Green Color

Application in a standard soft-drink (11.0° B—pH=3.0 made of saccharose, citric acid, potassium sorbate and sodium benzoate as preservatives) of a green emulsion (ColorFruit® Green 801 WSS) at 0.3, 0.33 or 0.36 g/L drink and combined with 0.6 g/L of cloudifier (white emulsion used to increase the cloudiness).

2-A Colorimetric values measured with existing spectrocolorimeter Datacolor 650:

TABLE 4
Colorimetric values measured with Datacolor 650.

| Color dosage | | | | |
|---|---|---|---|---|
| in g/L | L* | C* | h | DE2000 |
| 0.30 | 31.51 | 27.99 | 126.36 | REF. |
| 0.33 | 29.95 | 28.21 | 127.83 | 1.31 |
| 0.36 | 28.71 | 28.34 | 128.93 | 2.34 |

2-B Colorimetric values measured according to the present invention:

TABLE 5
Colorimetric values measured according
to the invention.

| Color dosage | | | | |
|---|---|---|---|---|
| in g/L | L* | C* | h | DE2000 |
| 0.30 | 72.51 | 52.84 | 151.09 | REF. |
| 0.33 | 69.76 | 54.45 | 150.77 | 2.15 |
| 0.36 | 67.38 | 54.36 | 151.69 | 4.00 |

DE2000 is calculated to indicate the difference of color between 2 samples.

The experiment showed that the new method and set-up was more sensitive and was able to detect differences more easily. Lightness and chroma values were much higher with the new method corresponding to a vivid color.

All 3 values L*, C* and h obtained with the old method were significantly lower than with the new method. The values obtained with Datacolor giving L* and C* values around 30 corresponded to a very dark and dull shade.

The virtual color, calculated from the colorimetric values, given by the software of the old method, was systematically duller and darker than the color visually observed on the sample measured on.

The color read out, the virtual color represented by the software that was based on the L* C* and h, from the new set-up showed the same color as visually observed on the sample measured on.

### Example 3

### Color Measurement of Liquid with Pink Color

Application in a standard soft-drink (11.0° B—pH=3.0 made of saccharose, citric acid, potassium sorbate and sodium benzoate as preservatives) of a pink cloudy emulsion (Vegex® Red 500 WS) at 0.120, 0.132 or 0.144 g/L drink and combined with 0.6 g/L of cloudifier (white emulsion used to increase the cloudiness).

3-A Colorimetric values measured with existing spectrocolorimeter Datacolor 650:

TABLE 6
Colorimetric values measured with Datacolor 650.

| Color dosage in g/L | L* | C* | h | DE2000 |
|---|---|---|---|---|
| 0.120 | 33.92 | 34.48 | 28.07 | REF. |
| 0.132 | 33.12 | 35.73 | 27.54 | 0.84 |
| 0.144 | 32.40 | 37.09 | 26.64 | 1.70 |

3-B Colorimetric values measured according to the present invention:

TABLE 7
Colorimetric values measured with the invention.

| Color dosage in g/L | L* | C* | h | DE2000 |
|---|---|---|---|---|
| 0.120 | 76.24 | 55.11 | 7.35 | REF. |
| 0.132 | 74.72 | 58.34 | 7.28 | 1.43 |
| 0.144 | 73.08 | 61.32 | 7.63 | 2.88 |

DE2000 is calculated to indicate the difference of color between 2 samples.

Lightness and chroma values were much higher with the new method corresponding to a vivid color as it is in reality.

This experiment showed that the new method and set-up was more sensitive and was able to detect differences more easily. The hue value obtained with the new method (around 7.0) corresponded to a pink shade whereas the hue obtained with the spectrocolorimeter corresponded to a redder color.

L*and C* values obtained with the old method were significantly lower than with the new method and invention. The values obtained with Datacolor giving a L* and C* values between 32.4 and 37.09 corresponds to a very dark and dull shade.

The virtual color, calculated from the colorimetric values, given by the software of the old method, was systematically more dull and darker than the color visually observed on the sample measured on.

The virtual color represented by the software that was based on the L* C* and h from the new set-up showed the same color as visually observed on the sample measured on.

### Example 4

### Color Measurement of Liquid with Orange Color

Application in a standard soft-drink (11.0° B—pH=3.0 made of saccharose, citric acid, potassium sorbate and sodium benzoate as preservatives) of an orange cloudy emulsion (Vegex® Orange Red 400 WSS) at 0.20, 0.22 or 0.23 g/L drink.

4-A Colorimetric values measured with existing spectrocolorimeter Datacolor 650:

TABLE 8
Colorimetric values measured with Datacolor 650.

| Color dosage in g/L | L* | C* | h | DE2000 |
|---|---|---|---|---|
| 0.20 | 5.56 | 24.80 | 22.59 | REF. |
| 0.22 | 4.84 | 22.34 | 21.80 | 1.36 |
| 0.23 | 4.15 | 19.68 | 21.23 | 2.91 |

4-B Colorimetric values measured according to the present invention:

TABLE 9
Colorimetric values measured with the invention.

| Color dosage in g/L | L* | C* | h | DE2000 |
|---|---|---|---|---|
| 0.20 | 72.40 | 98.72 | 55.51 | REF. |
| 0.22 | 71.54 | 104.41 | 55.98 | 1.29 |
| 0.23 | 69.53 | 106.00 | 55.64 | 2.56 |

DE2000 was calculated to indicate the difference of color between 2 samples.

The lightness measured with the colorimeter showed that the light cannot go through the product and the optical fiber connected to the entrance slit of the detector received almost no signal. It corresponded to a black color. The hue measured corresponded to a red shade and not an orange shade.

The experiment showed that the present invention was more sensitive and was able to detect differences more easily. The hue measured corresponded to an orange color.

L*, C* and h values obtained with the old method were significantly lower than with the new method and invention.

The lightness L* measured between 4.15 and 5.56 corresponded to black.

C* values or intensity measured below 23.0 corresponded to a very dull shade whereas the color shade of the sample or the color observed on the sample measured is a vivid orange shade.

The hue measured between 21.23 and 22.59 corresponded to a red shade and not an orange shade.

The virtual color, calculated from the colorimetric values, given by the software of the old method was systematically more dull and darker than the color visually observed on the sample measured on.

The virtual color represented by the software as a color read out that was based on the L* C* and h from the new set-up showed the same color as visually observed on the sample measured on.

1. A set-up for measuring a color of a turbid liquid to obtain a result matching a visual color observation comprising:
i) a housing wherein at least 50% of the inside area is white; and
ii) a light source illuminating the inside of the housing giving consistent, light conditions;
iii) a transparent sample holder placed inside the housing positioned against the optical fiber connected to the entrance slit of the detector of a spectrophotometer;
iv) a colored turbid liquid to be poured into the sample holder of iii);
v) a spectrophotometer for measuring the color spectrum of the colored turbid liquid;
vi) a computer; and
vii) a spectrophotometric software which is able to collect and calculate colorimetric data.

2. The set-up according to claim 1, further comprising a spectrophotometer, wherein:
the light source mimics daylight;
the transparent sample holder contains a stationary, turbid liquid;
and
the colorimetric data are lightness, chroma and hue.

3. The set-up according to claim 1, wherein the colorimetric data are lightness, chroma and hue.

4. The set-up according to claim 1, wherein the light source of ii) is mimicking daylight.

5. The set-up according to claim 1, wherein at least 65% of the inside area of the housing is colored white.

6. (canceled)

7. The set-up according to claim 1, wherein the sample holder is colorless.

8-11 (canceled)

12. The set-up according to claim 1, wherein the sample holder is removable.

13. The set-up according to claim 1, wherein the sample holder is the packaging of a final product.

14. The set-up according to claim 1, wherein the turbid liquid is a non-diluted final product.

15. (canceled)

16. A method for measuring the color of a turbid liquid matching a visual color observation, comprising:
i) providing a housing wherein at least 50% of the inside area is white, the housing comprising a light source; a spectrophotometer; and a transparent sample holder containing a turbid liquid sample;
ii) illuminating the turbid liquid sample with the the light source;
iii) measuring the color spectrum of the turbid liquid sample with the spectrophotometer;
iv) converting the measured color spectrum into colorimetric values using a computer and a software for doing so; and
v) providing a digital color read out corresponding to
a) a visual observation of the color of the turbid liquid or
b) the color of the turbid liquid.

17. A method for measuring the color of a final product comprising a turbid liquid matching a visual color observation, comprising:
i) providing a housing wherein at least 50% of the inside area is white, the housing comprising a light source; a spectrophotometer; and a final product comprising a turbid liquid;
ii) illuminating the turbid liquid with the light source;
iii) measuring the color spectrum of the turbid liquid with the spectrophotometer;
iv) converting the measured color spectrum into colorimetric values using a computer and a software for doing so; and
vi) providing a digital color read out corresponding to
a) a visual observation of the color of the turbid liquid, or
b) the color of the turbid liquid.

18. The method of claim 17, wherein the final product is a beverage placed in transparent bottle.

19. The method according to claim 16 for measuring the color of a turbid liquid in the form of chroma, hue and lightness matching a visual color observation: wherein the light source mimics daylight and the converting comprises
converting the measured color spectrum into spectrocolorimetric values using a computer and a software for doing so; and
the digital color read out is in the form of chroma, hue and lightness corresponding to
a) a visual observation, or
b) the color of the turbid liquid

20. The method according to claim 16, wherein at least 65% of the inside area of the housing is white.

21. (canceled)

22. The method according to claim 16, wherein the transparent sample holder is the packaging of a final product.

23. The method according to claim 16, wherein the transparent sample holder is a final product.

24. The method according to claim 16, wherein the liquid is stationary.

25. (canceled)

26. The method according to claim 17, wherein the product is selected from food products, paints, hair products, body care products and cosmetics.

27. The method according to claim 26, wherein the food product is selected from a dairy product, an oil product and a beverage.

28. The method according to claim 27, wherein the beverage is selected from fruit juice products, smoothies, soft drinks, wine and beer.

29-30 (canceled)
